(19) United States
(12) Patent Application Publication
(10) Pub. No.: US 2011/0159059 A1
(43) Pub. Date: Jun. 30, 2011

(54) Oral Care Beadlets

(75) Inventor: Vinayak B. Randive, Thane (IN)

(73) Assignee: FMC Corporation, Philadelphia, PA (US)

(21) Appl. No.: 12/967,774

(22) Filed: Dec. 14, 2010

(30) Foreign Application Priority Data
Dec. 28, 2009 (IN) 2714/DEL/2009

(51) Int. Cl. A61K 8/11; A61K 8/73; A61Q 11/00

(52) U.S. Cl. 424/401; 424/49

(57) ABSTRACT

A single unit oral care product comprises an oral care composition comprising a polysaccharide, a secondary polymer, and a sequestrant where an outer surface of the oral care composition is encapsulated by an outer coating comprising the polysaccharide in cross-linked form. The single unit oral care product may be made by combining the gellable polysaccharide, the secondary polymer, and the sequestrant to form an oral care composition and forming at least a portion of the oral care composition into a shaped unit. The shaped unit is then immersed in an aqueous solution of a divalent salt to form a gelled oral care composition. Optionally, the gelled oral care composition may be washed with a second sequestrant.

### FIELD OF THE INVENTION

The invention relates to oral care beadlet compositions and methods of making the same.

### BACKGROUND OF THE INVENTION

Historically, toothpaste was first introduced commercially as a cream in bottles or jars. Subsequent evolution in tube packaging, from aluminum tubes to modern laminated tubes, made toothpaste usage more consumer friendly. Although tube packaging is popular for several reasons, there are some disadvantages. In particular, tube packaging has a higher cost, for example due to maintenance of high cost packaging lines, which adds to the cost of the final product. Also, consumers may have problems with the tubes, such as difficulty in squeezing out the last portions of toothpaste, dropping/losing caps, and messiness around the caps. Additionally, tubed toothpaste is not as convenient for travel.

### SUMMARY OF THE INVENTION

The oral care compositions of the present invention are gelled on the surface to form a single unit oral care beadlet. Aspects of the present invention include the oral care beadlet compositions and methods of making the same.

According to an embodiment of the present invention, a single unit oral care product comprises an oral care composition comprising a polysaccharide, a secondary polymer, and a sequestrant, wherein an outer surface of the oral care composition is encapsulated by an outer coating comprising the polysaccharide in crosslinked form.

According to another embodiment of the present invention, the single unit oral care product is made by combining a gellable polysaccharide, the secondary polymer, and the sequestrant to form an oral care composition and forming at least a portion of the oral care composition into a shaped unit. The shaped unit is then immersed in an aqueous solution of a divalent salt to form a gelled oral care composition. Optionally, the gelled oral care composition may be washed with a second sequestrant.

### DETAILED DESCRIPTION OF THE INVENTION

Because the oral care compositions of the present invention are gelled on the surface to form a single unit oral care beadlet, the oral care beadlets in the present invention offer several benefits over a tubed oral care product. For example, each oral care beadlet is a single measured unit dose. The beadlets may be packaged in smaller, simpler containers eliminating the need for maintaining expensive tube packaging lines. The smaller packaging makes the beadlets easier to carry, and the unique beadlet dosages makes it easier for the consumer to ascertain when refills are needed.

As used herein, “beads” or “beadlets” are used to describe the state or form of the oral care composition when the surface is gelled. The beads or beadlets may be of any suitable shape or size. For example, a beadlet may be a single use portion size of about 0.5 to 1 gram and may be approximately spherical in shape. It is envisioned, however, that the beadlets may be larger or smaller and may be formed into any convenient shape, e.g., oval, etc.

As used herein, “syneresis” is a term used by those skilled in the art to describe separation of liquid components in a formulation. Syneresis is considered a severe form of instability in a formulation, and is usually regarded to be unsuitable. A slight indication of syneresis in beads occurs when the beads acquire an oily appearance on the surface. A severe form of syneresis occurs when the oil separates out and settles to the bottom, for example, of the jar holding the beads. An effect of syneresis may include the flavor separating out or being non-uniformly distributed in the beadlet. The gelled oral care composition of the present invention preferably exhibits little to no syneresis.

As used herein, unless specified otherwise, the values of the constituents or components of the compositions are expressed in weight percent or % by weight of each ingredient in the composition.

In one embodiment of the invention, a single unit oral care product comprises an oral care composition comprising a polysaccharide, a secondary polymer, and a sequestrant. An outer surface of the oral care composition is encapsulated by an outer coating comprising the polysaccharide in crosslinked form.

Any suitable polysaccharide may be used in the present invention. Most preferably, the polysaccharide is a gellable polysaccharide. As used herein, “gellable” or “gelled” is meant to encompass the ability for the polysaccharide to gel (i.e., gel forming), for example, through crosslinking. Gellable polysaccharides may include, for example, alginates, carrageenans, chitosans, pectins, and mixtures thereof. In a preferred embodiment, the gellable polysaccharide is an alginate. Alginates are salts of alginic acid. Alginic acid, which is isolated from seaweed, is a polyuronic acid made up of two uronic acids: D-mannuronic acid (M) and L-guluronic acid (G). Alginates are not random copolymers, but consist of blocks of similar and alternating residues, for example, MMMM, GGGG, and GMGM, and are generally useful in the form of alginic acid or salts thereof. The ratio of mannuronic acid and guluronic acid varies with factors such as seaweed species, plant age, and part of the seaweed (e.g., stem, leaf). The G content and molecular weight of the alginate are not particularly limited. Preferred alginates are gellable alginates having a high affinity for bivalent cations, moderate gelation, good dispersibility when manually deformed, and are odorless and tasteless. Suitable alginates include sodium alginates available under the tradename PROTANAL® obtainable from FMC Corporation with headquarters in Philadelphia, Pa. Suitable amounts of polysaccharide may range from about 0.1 to 10% by weight of the composition, preferably about 0.5 to 3% by weight of the composition, more preferably about 1%.

The oral care composition comprises a secondary polymer. The secondary polymer may be used to control syneresis and texture of the oral care composition. Without wishing to be bound to a particular theory, the secondary polymer may also help to control the wall thickness of gelation and the internal viscosity of the beadlet. In an exemplary embodiment, the secondary polymer is a non-gellable polysaccharide such as a cellulose or cellulose derivative, e.g., an alkali metal carboxymethylcellulose. In a preferred embodiment, the secondary polymer is sodium carboxymethylcellulose or potassium carboxymethylcellulose. Most preferably, the secondary polymer is sodium carboxymethylcellulose. The alkali metal carboxymethylcellulose may, for example, have a degree of substitution of from about 0.6 to about 1.5, or from about 0.7 to about 1.5, or from about 0.7 to about 1.2. Suitable amounts of the secondary polymer may range from about 0.1 to 10% by weight of the composition, preferably about 0.5 to 2% by weight of the composition, more preferably about 0.8 to 1%.

The oral care composition further comprises a sequestrant. Sequestrants are compounds that bind or complex the gelling ion, e.g., calcium, from the surface of the beadlet. In other words, sequestrants act as a chelating agent to form chelate complexes with polyvalent metal ions. In typical applications, sequestrants are used as a rinsing/washing agent on the surface of a previously gelled polysaccharide. It was discovered in the present invention that including the sequestrant in the oral care composition provided improved texture and softness in the gelled beadlet.

Suitable sequestrants may include, for example, phosphate and phosphate salts (e.g., sodium hexametaphosphate, tetrasodium pyrophosphate, disodium hydrogen orthophosphate), polycarboxylic acids (including hydroxy-substituted polycarboxylic acids) and salts thereof (e.g., sodium citrate), as well as polyaminocarboxylic acids and salts thereof (e.g., ethylenediaminetetraacetic acid (EDTA) and its salts). The sequestrant may, for example, contain two or more functional groups per molecule selected from the group consisting of phosphorus-containing acid groups, carboxylic acid groups, and salts thereof, particularly alkali metal salts thereof. The sequestrant may also be a substance containing at least one functional group per molecule selected from the group consisting of phosphorus-containing acid groups, carboxylic acid groups and salts thereof and at least one additional functional group selected from the group consisting of hydroxy groups and amine groups. In an exemplary embodiment, the sequestrant is a pyrophosphate. In a preferred embodiment, the sequestrant is tetrasodium pyrophosphate (TSPP). The sequestrant may be present, for example, in an amount of about 0.2 to 0.6% by weight of the oral care composition, although smaller or greater amounts could also be suitable depending upon the particular oral care composition and the particular method by which the beadlets are prepared using the composition. More specifically, more sequestrant may be added to the composition when the oral care composition is not later washed by a secondary sequestrant. If the oral care composition is washed by a second sequestrant, then less sequestrant is necessary in the oral care composition. Thus, if the beadlet is not washed with a secondary sequestrant, the sequestrant may be present in an amount of about 0.4 to 0.6%, or more preferably about 0.5% by weight of the oral care composition. If the beadlet is washed with a secondary sequestrant, the sequestrant may be present in an amount of about 0.2 to 0.4%, or more preferably about 0.25% by weight of the oral care composition. The secondary sequestrant wash may be of any suitable concentration. For example, the secondary sequestrant wash may be about 0.25% to 1.0% (preferably about 0.25%) of the secondary sequestrant in water.

The ingredients in the oral care composition may be present in any suitable amounts as would be ascertainable by one skilled in the art. In an exemplary embodiment, the secondary polymer and the polysaccharide are present in a weight ratio of about 0.8:1 to 1:1. As would be recognized by one skilled in the art, each of the ingredients should be suitable for use as a toothpaste, i.e., not harmful if consumed. The composition should be formulated to have a suitable consistency for the application. In particular, the oral composition, prior to being gelled, may have a viscous consistency, e.g., similar to toothpaste. Water, for example, distilled or deionized water, may also be added to the composition, e.g., to adjust the consistency.

In an exemplary embodiment, the oral care composition does not include calcium in any form. For example, toothpaste compositions may generally comprise calcium phosphate, calcium sodium phosphosilicate, etc. as ingredients, but in an embodiment of the present invention, the oral care composition does not comprise calcium.

The oral care composition may be formed using any suitable techniques known in the art, e.g., mixing/blending techniques commonly used for producing toothpaste. Once the oral care composition is prepared, it is formed into small portions (e.g., beadlets). The small portions are gelled on the outer surface. Thus, the oral care composition is at least partially if not fully encapsulated by a crosslinked outer coating of the polysaccharide. For example, the surface of the oral care composition may be gelled by allowing the gellable polysaccharide in the oral care composition to crosslink, for example, with a divalent cation. Suitable divalent cations may include, for example, calcium(2+), barium(2+), strontium(2+), iron(2+), zinc(2+), copper(2+), magnesium(2+) and mixtures thereof. In an exemplary embodiment, the crosslinked outer coating has occurred because the polysaccharide, e.g., alginate, has been crosslinked with a divalent cation. In a preferred embodiment, the divalent cation is calcium or zinc. Most preferably, the divalent cation is calcium.

Gelation may be used as a measure of the stickiness, sturdiness, and/or hardness of the beads. The beads should be adequately gelled so that the outer shell is not sticky or tacky and can withstand at least some deformation of the bead (e.g., a strong shell prevents deformation in packaging and storage conditions). The shell also provides a definite shape to the bead. However, a balance must be struck between adequate gelation and over gelation. Over gelation occurs with the excessive interaction of the polysaccharide in the formulation with the divalent ions present in the outer shell (after the beadlets are treated in an aqueous divalent salt bath). Over time, the degree of interaction between these ions increases which leads to a progressively thickening shell. When this happens, the shell is difficult to break and even more difficult to disperse. After chewing orally, the shell breaks up into lumps which cannot be dispersed even while brushing. The compositions disclosed herein, however, have been found to be capable of striking the appropriate balance of gelation without being too sticky or too over-gelled.

Additional ingredients may optionally be included in the oral care composition, for example, humectants, flavors, foaming agents, sweeteners, coloring agents, preservatives, abrasives, thickeners, and/or active ingredients. In an exemplary embodiment, the oral care composition further comprises at least one additional ingredient selected from the group consisting of a secondary hydrocolloid, flavors, foaming agents, salts, sweeteners, preservatives, surfactants, coloring agents, abrasives, thickeners, active agents, and mixtures thereof.

Any suitable humectant known to those skilled in the art may be included in the oral care composition. Humectants help to plasticize and retain moisture in the composition and to stabilize the texture of the beadlet shell. Humectants also retain water and provide desired texture to toothpastes. In an exemplary embodiment, the humectant is selected from the group consisting of glycerin, sorbitol, polyoxyethylene glycol (PEG), xylitol, and mixtures thereof. In particular, suitable liquid PEGs are in the low molecular weight range 200 to 600. In a preferred embodiment, the oral care composition comprises sorbitol and/or xylitol. Xylitol is a superior toothpaste humectant that boosts the cavity fighting power of fluorides.

Other additives such as solubilizers, flavors, foaming agents, sweeteners, coloring agents, preservatives, thickeners, abrasives, and active agents may also be added. Solubilizers may increase the solubility of a substance, such as solubilizing generally water-insoluble components in the oral care composition (e.g., flavoring oils). Solubilizers may be ionic or nonionic. Any suitable solubilizer may be used, including castor oil, ethoxylated fatty acid glycerides, ethoxylated fatty acid sorbitan partial esters, or fatty acid partial esters of glycerol and sorbitan ethoxylates, and most preferably hydrogenated ethoxylated castor oil. Flavors, such as mint, may be in the form of flavor oils. The flavors are added to impart a desirable taste sensation, to freshen breath, and/or to mask the taste of foaming agents. Foaming agents may be added to create the “foaming action” consumers associate with toothpastes. The foaming agents may help assist with the removal of biofilm and food particles during brushing. Any suitable foaming agent may be used. Sweeteners may be added to the composition in order to modify the flavor profile. For example, artificial sweeteners, such as saccharin, may be added because they are less likely to promote cavity formation. Coloring agents may be added to the composition, such as dyes and pigments, in order to achieve a desired visual effect (color and/or opacity). Preservatives may be added, for example, to prevent the growth of microorganisms. Common preservatives include sodium benzoate, methyl paraben, and ethyl paraben. Thickeners may be added to provide suitable stable rheology. Examples of thickeners include, for example, colloidal silica, carrageennan, carboxy methyl cellulose, xanthan. Abrasives may also be added to the composition. Abrasives are added into toothpastes to polish the teeth with a minimum level of abrasive action and to remove plaque, food debris, and superficial stains. In one embodiment, more than one abrasive may be used, for example, silica as a primary abrasive. In a preferred embodiment, silica abrasives are used. The abrasive selected must be compatible with the other ingredients. Active agents may also be added to the oral care composition. Typical active agents include, for example, fluoride compounds, antimicrobial enzymes, and astringents.

The gelled oral care composition beadlets described herein were found to have minimal syneresis and good texture. Thus, the single unit beadlets provide a chewable oral product that may be used by the consumer to deliver a toothpaste-like composition for brushing.

In another embodiment of the present invention, a method of making a single unit oral care product comprises combining a gellable polysaccharide, a secondary polymer, and a sequestrant to form an oral care composition. At least a portion of the oral care composition is then formed into a shaped unit. The shaped unit is then immersed in an aqueous solution of a divalent salt to form a gelled oral care composition.

The ingredients for the oral care composition may be combined in any sequence or simultaneously. In a preferred embodiment, the gums (e.g., polysaccharides) are mixed with the humectants. Water is then added and the mixture is stirred. The sequestrant, e.g., TSPP, is added to the mixture along with additional components such as a sweetener and/or a preservative with continued stirring. The composition is then mixed with an abrasive, such as silica, under vacuum conditions (e.g., using a vacuum mixer). It was discovered that air in the composition can produce shell weakness in the resulting beadlet and/or flavor separation, which can adversely affect the quality of the toothpaste. Thus, in a preferred embodiment, the oral care composition is deaerated. In other words, a vacuum is applied using any suitable equipment to remove all or essentially all of the air from the composition prior to gelling. Additional ingredients, such as flavor and surfactant, may also be added in the vacuum mixer. The composition may have the viscous consistency, e.g., of toothpaste.

At least a portion of the oral care composition is then formed into a shaped unit. A portion of the oral care composition may formed into a single unit dose using any suitable techniques known in the art. For example, a single portion may be metered out or equal-sized bead portions may be cut. The portions of oral care composition may be shaped into any suitable size and shape, e.g., spherical, oval, etc., using any techniques or equipment known to those skilled in the art.

The shaped unit is then immersed in an aqueous solution of a divalent salt to form a gelled oral care composition. The aqueous divalent salt solution may comprise water or any other aqueous solvent. The divalent salt may be dissolved in the aqueous solution using any standard mixing techniques. The divalent salt may include any water-soluble salts comprising a divalent ion, such as Ca²⁺, Ba²⁺, Sr²⁺, Zn²⁺, Fe²⁺, Mg²⁺ and Cu²⁺. The anionic portion of the salt may be, for example, a halide, sulfate, carboxylate, phosphate, nitrate, carbonate or the like. In an exemplary embodiment, the divalent salt is selected from the group consisting of calcium chloride, zinc lactate, and zinc carbonate. In a preferred embodiment, the divalent salt is calcium chloride. Treatment baths may be prepared by dissolving salts in deionized water. For example, a 5% calcium bath may be prepared by dissolving fused calcium chloride in water. A saturated solution of 5% zinc lactate bath may prepared by adding zinc lactate to deionized water and heating, for example, to 75° C. for 10 minutes.

The shaped unit may be dropped directly into a bath comprising the aqueous divalent salt solution. The shaped units may remain submerged for a certain period of time and under certain conditions to allow the surface of the shaped unit to gel (e.g., the polysaccharide in the composition crosslinks with the divalent cation). Thus, the outer surface of the oral care composition is encapsulated by a gel membrane. The gelled oral care composition is then removed from the bath. The gelled oral care composition may be rinsed with water after being removed from the salt bath and/or may be dried.

In an exemplary embodiment, the gelled oral care composition is then washed with a second sequestrant. The second sequestrant may be the same or different than the primary sequestrant in the composition. Suitable second sequestrants may include, for example, phosphate salts, sodium citrate, ethylenediaminetetraacetic acid (EDTA) and salts thereof. In an exemplary embodiment, the second sequestrant is also tetrasodium pyrophosphate (TSPP). The gelled oral care composition may be washed using any suitable techniques known in the art, e.g., immersed in a bath of the sequestrant. Treatment baths may be prepared by dissolving salts in deionized water, e.g., a 0.25%, 0.5%, or 1.0% TSPP bath. The sequestrant may bind or complex the gelling ion, e.g., calcium, in the gelled wall/membrane, thus changing the physical characteristics of the surface, for example, making the beadlet more water-soluble. In other words, sequestrants act as a chelating agent to form chelate complexes with the polyvalent metal ions.

As previously discussed, if the oral care composition is washed by a second sequestrant, less sequestrant may be necessary in the oral care composition. For example, when the beadlet is washed with a secondary sequestrant, the sequestrant may be present in an amount of about 0.2 to 0.4%, or more preferably about 0.25% by weight of the oral care composition. The secondary sequestrant wash may have a concentration of about 0.1 to 1% by weight of the second sequestrant in an aqueous bath. In an exemplary embodiment, the second sequestrant is present in an amount of about 0.25% to 1.0% (preferably about 0.25%) of the secondary sequestrant in water.

The gelled oral care composition may be washed with water prior to and/or subsequent to being washed with the second sequestrant. The beadlets may also be rinsed multiple times, e.g., about three times, by immersion in water. The gelled oral care composition may also be dried using any well known techniques at any step during the process and is preferably dried subsequent to washing with the second sequestrant.

In an exemplary embodiment, an oral care composition comprising an alginate, a sodium carboxymethylcellulose, and a first portion of tetrasodium pyrophosphate, wherein an outer surface of the oral care composition is encapsulated by an outer coating comprising the alginate crosslinked with a calcium cation, wherein the outer coating has been contacted with a second portion of tetrasodium pyrophosphate.

It will be recognized that the weight percents of the ingredients in the beadlet composition may be adjusted accordingly to maintain the desired results, such as degree of gelling. Such routine adjustment of the composition is fully within the capabilities of one having skill in the art and is within the scope and intent of the present invention.

### EXAMPLES

The acceptability of the beads was assessed using the three parameters: gelation, texture, and syneresis. Gelation was rated by the softness of the gelled bead and by flexure which is primarily due to the outer shell characteristics. Internal gelation, if present, contributed to this parameter. Gelation was rated on the following scale:

### 1—Very Good, Soft Gelling

### 2—Soft Gelling

### 3—Moderate Gelling

### 4—Semi Hard, Not Acceptable

### 5—Hard, Not Acceptable

### 1-3 (Acceptable); 4-5 (Unacceptable)

Texture was rated based on the bead characteristics, which were experienced while chewing the bead in the oral cavity. Outer texture would include parameters such as a smooth feel to the bead, while inner texture would include softness and ease of chewability. The ideal texture would include a bead with a smooth, outer shell, that when broken yields a soft pasty inner core with no lumps. The textures of the beads were tested both on the basis of manual breaking by hand as well as oral chewing. The average of the two observations was considered. Texture was rated on the following scale:

### 1—Very Smooth

### 2—Smooth

### 3—Slightly Hard

4—Hard, Not dispersible

### 5—Very Hard, Not Acceptable

### 1-3 (Acceptable); 4-5 (Unacceptable)

Syneresis was considered based on its severity. A slight indication of syneresis in the beads occurred when the beads acquired an oily appearance on the surface. A severe form of syneresis occurred when the oil separated out and settled to the bottom of the jar holding the beads.
1—No Syneresis, acceptable
2—Little syneresis, acceptable
3—Syneresis, acceptable
4—Heavy Syneresis, NOT acceptable
5—Severe Syneresis, NOT acceptable

### 1-3 (Acceptable); 4-5 (Unacceptable)

### Example 1

Example 1 compares three formulations according to embodiments of the present invention in Table 1 with varying amounts of sequestrant, TSPP, in an oral care formulation. The formulation includes as the gellable polysaccharide, an alginate, PROTANAL® LF 200S available from FMC BioPolymer, with headquarters in Philadelphia, Pa. The secondary polymer is CMC under the tradename AQUALON® cellulose gum 9M31FD available from Ashland Aqualon Functional Ingredients, Wilmington Del. The sequestrant is tetra sodium pyrophosphate (TSPP) anhydrous extra pure. Sorbitol and PEG are humectants. PEG 300 is obtainable from Merck Specialties Pvt. Ltd, with offices in Mumbai, Maharashtra, India. Saccharin is a sweetener, and sodium benzoate is a preservative. CREMOPHOR RH40 is a hydrogenated ethoxylated castor oil available from BASF India Ltd. with offices in Mumbai, India. Castor oil derivatives may be used as a solubilizer for flavoring oils in toothpaste. ZEODENT® 113 and ZEODENT® 165 are different grades of silica, an abrasive and a thickener, respectively, which are available from J. M. Huber, with offices in Havre de Grace, Md. The flavor is a toothpaste flavor, which is lab-made. SLS, sodium lauryl sulphate, is a surfactant.

TABLE 1

| | Formulation | | |
|---|---|---|---|
| | A | B | C |
| PROTANAL 200S | 1.00 | 1.00 | 1.00 |
| CMC 9M31FD | 0.80 | 0.80 | 0.80 |
| TSPP | 0.25 | 0.50 | 1.00 |
| Sorbitol | 60.00 | 60.00 | 60.00 |
| PEG-300 | 2.00 | 2.00 | 2.00 |
| Saccharin | 0.20 | 0.20 | 0.20 |
| Sodium Benzoate | 0.20 | 0.20 | 0.20 |
| CREMOPHOR | 5.00 | 5.00 | 5.00 |
| RH40 | | | |
| ZEODENT-165 | 7.00 | 7.00 | 7.00 |
| ZEODENT-113 | 11.00 | 11.00 | 11.00 |
| Flavour | 0.90 | 0.90 | 0.90 |
| SLS | 2.00 | 2.00 | 2.00 |
| Water | 9.65 | 9.40 | 8.90 |
| Total | 100.00 | 100.00 | 100.00 |
| Calcium Bath | 5% | 5% | 5% |
| Cure Time | 5 mins | 5 mins | 5 mins |
| Water Washing | 3 | 3 | 3 |
| Time | | | |

The treatment baths were prepared by dissolving salts in deionized water. The 5% calcium bath was prepared by adding 50 grams of fused calcium chloride in 950 grams of water

Oral care beads were formed by cutting equal sized portions of toothpaste and dropping the toothpaste portions directly into the calcium bath. The beads were submerged in the bath for the prescribed time after which the beads were removed with draining to remove salt solution and then rinsed three times by immersing in fresh tap water for the prescribed time followed by draining to remove rinse water. The beads were laid on the laboratory bench top to air dry after the final rinse step.

Formulation A resulted in a soft bead with a thin shell. The bead had uniform gelling, but the shell was not dispersible. The ratings were 3 for gelation, 3 for texture, and 1 for syneresis. Formulation B resulted in a soft bead with a thin shell and had ratings of 2 for gelation, 1 for texture, and 1 for syneresis. Formulation C resulted in a bead with a thin shell, but exhibited 80% breakage during drying.

### Example 2

Example 2 compares formulations according to embodiments of the present invention as shown in Table 2 with some formulations undergoing a wash with a second sequestrant, TSPP.

TABLE 2

| Formulation | D | E | F | G | H | I |
|---|---|---|---|---|---|---|
| PROTANAL 200S | 1.00 | 1.00 | 1.00 | 1.00 | 1.00 | 1.00 |
| CMC 9M31FD | 1.00 | 1.00 | 1.00 | 0.80 | 0.80 | 0.80 |
| TSPP | 0.25 | 0.25 | 0.25 | 0.25 | 0.25 | 0.25 |
| Sorbitol | 60.00 | 60.00 | 60.00 | 60.00 | 60.00 | 60.00 |
| PEG - 300 | 2.00 | 2.00 | 2.00 | 2.00 | 2.00 | 2.00 |
| Saccharin | 0.20 | 0.20 | 0.20 | 0.20 | 0.20 | 0.20 |
| Sod Benzoate | 0.20 | 0.20 | 0.20 | 0.20 | 0.20 | 0.20 |
| CREMOPHOR RH40 | 5.00 | 5.00 | 5.00 | 5.00 | 5.00 | 5.00 |
| ZEODENT- 165 | 7.00 | 7.00 | 7.00 | 7.00 | 7.00 | 7.00 |
| ZEODENT- 113 | 11.00 | 11.00 | 11.00 | 11.00 | 11.00 | 11.00 |
| Flavour | 0.90 | 0.90 | 0.90 | 0.90 | 0.90 | 0.90 |
| SLS | 2.00 | 2.00 | 2.00 | 2.00 | 2.00 | 2.00 |
| Water | 9.45 | 9.45 | 9.45 | 9.65 | 9.65 | 9.65 |
| Total | 100.00 | 100.00 | 100.00 | 100.00 | 100.00 | 100.00 |
| Calcium Bath | 5% | 5% | 5% | 5% | 5% | 5% |
| Cure Time | 5 mins | 5 mins | 5 mins | 5 mins | 5 mins | 5 mins |
| Water Washing Time | 3 | 3 | 3 | 3 | 3 | 3 |
| 1% TSPP Bath | — | — | — | — | 1 mins | |
| 0.5% TSPP Bath | — | 1 min | — | — | | 1 mins |
| 0.25% TSPP Bath | — | — | 1 min | | | |
| Water Washing Time | | 3 | 3 | | 3 | 3 |

The oral care beads were prepared as explained in Example 1. Where indicated, the beads were further treated in treatment baths containing a chelating agent (TSPP) by adding the treatment solution to the drained beads and submerging the beads in the treatment solution for the prescribed time, removing the beads from the treatment solution and then rinsing three times by immersing in fresh tap water each time followed by draining to remove the rinse water. The beads were laid on the laboratory bench top to air dry after the final rinse step.

Formulation D resulted in a thin soft shell with a soft core with ratings of 3.5 for gelation, 3 for texture, and 4 for syneresis. Formulation E had a thin soft shell with a soft core and had ratings of 2 for gelation, 2 for texture, and 3 for syneresis. Formulation F exhibited a thin shell with a soft shell and core. The ratings were 2 for gelation, 2 for texture, and 3 for syneresis. Formulation G had a thin shell and a soft bead. While the gelling was uniform, the shell was not dispersible. It had ratings of 3 for gelation, 3 for texture, and 1 for syneresis. Formulation H was a soft bead having a thin shell with ratings of 2 for gelation, 1 for texture, and 1 for syneresis. Formulation I was a soft bead with a thin shell having ratings of 2 for gelation, 1 for texture, and 1 for syneresis. Thus, the formulations undergoing a wash with a second sequestrant (Formulation E, F, H, and I) exhibited better characteristics for gelation, texture and syneresis then the formulations without such a wash (Formulations D and G).

### Example 3

Example 3 compares different formulations according to embodiments of the present invention (Formulations K, L, M, O, P) to two comparative formulations lacking a secondary polymer, CMC (Formulations J and N). An additional ingredient in some of these compositions include SCOGIN HV, an alginate obtainable from FMC Corp.

TABLE 3

| Formulation | J | K | L | M | N | O | P |
|---|---|---|---|---|---|---|---|
| PROTANAL 200S | | 1.00 | 1.00 | 1.00 | — | | |
| CMC 9M31FD | | 1.00 | 1.00 | 0.80 | — | 0.50 | 0.60 |
| SCOGIN HV | | — | | | 2.00 | 1.10 | 1.00 |
| Glycerin | | 37.00 | — | — | 37.00 | 10.00 | — |
| Sorbitol | | 25.00 | 60.00 | 60.00 | 25.00 | 50.00 | 60.00 |
| PEG - 300 | | — | 2.00 | 2.00 | — | 2.00 | 2.00 |
| Saccharin | | 0.20 | 0.20 | 0.20 | 0.20 | 0.20 | 0.20 |
| Sod Benzoate | | 0.20 | 0.20 | 0.20 | 0.20 | 0.20 | 0.20 |
| TSPP | | 0.25 | 0.25 | 0.25 | 0.25 | 0.25 | 0.25 |
| CREMOPHOR RH40 | | 5.00 | 5.00 | 5.00 | 5.00 | 5.00 | 5.00 |
| ZEODENT- 165 | | 7.00 | 7.00 | 7.00 | 6.00 | 7.00 | 7.00 |
| ZEODENT- 113 | | 11.00 | 11.00 | 11.00 | 11.00 | 11.00 | 11.00 |
| Flavour | | 0.90 | 0.90 | 0.90 | 0.90 | 0.90 | 0.90 |
| SLS | | 2.00 | 2.00 | 2.00 | 2.00 | 2.00 | 2.00 |
| Water | | 9.45 | 9.45 | 9.65 | 10.45 | 9.85 | 9.85 |
| Total | 100.00 | 100.00 | 100.00 | 100.00 | 100.00 | 100.00 | 100.00 |
| Calcium Bath | 5% | 5% | 5% | 5% | 5% | 5% | 5% |
| Cure Time | 10 mins | 5 min | 5 mins | 5 mins | 5 mins | 5 mins | 5 mins |
| Water Washing Time | 3 | 3 | 3 | 3 | 3 | 3 | 3 |

The oral care beads were prepared as explained in Examples 1 and 2.

Control formulation J, without CMC (containing the following: Protanal 200S 2%; Glycerin 37%; sorbitol 25%; saccharin 0.2%; sodium benzoate 0.2%; TSPP 0.25%; Cremophor RH40 5%; Zeodent 165; Zeodent 113; flavor; SLS and water), was an entirely gelled, semi-hard shell having ratings of 5 for gelation, 5 for texture, and 1 for syneresis. Control formulation N was also entirely gelled and had ratings of 3 for gelation, 3 for texture, and 1 for syneresis.

Formulations K, L, M, O, and P each contained some CMC. Formulation

K exhibited a thin shell and soft beads, but showed 70% of breaking during preparation. Formulation K had ratings of 3.5 for gelation, 3 for texture, and 1 for syneresis. Formulation L had a thin shell with a soft shell and core and had ratings of 3.5 for gelation, 3 for texture, and 1 for syneresis. Formulation M had soft beads with uniform gelling, but the shell was not dispersible, and had ratings of 3 for gelation, 3 for texture, and 1 for syneresis. Formulation O exhibited a thin shell and soft beads with a rating of 2 for gelation, 2 for texture, and 1 for syneresis. Formulation P was also thin shelled and had soft beads with ratings of 3 for gelation, 2 for texture, and 1 for syneresis. Thus, the formulations including a secondary polymer, CMC, exhibited improved gelation, texture, and syneresis.

Formulation Q of the present invention was also prepared (Protanal S200 1.5%; CMC 0.75%; glycerin 37%; sorbitol 30%; saccharin 0.2%; sodium benzoate 0.2%; TSPP 0.25%; Zeodent 165 6%; Zeodent 113 11%; flavor 0.9%; SLS 2%; water 10.2%) and cured for 10 minutes in a calcium bath followed by a 3 minute water washing and was found to have the following ratings: gelation 3; texture 2; syneresis 1.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

What is claimed:

1. A single unit oral care product comprising:
an oral care composition comprising a polysaccharide, a secondary polymer, and a sequestrant,
wherein an outer surface of the oral care composition is encapsulated by an outer coating comprising the polysaccharide in crosslinked form.

2. A product according to claim 1, wherein the polysaccharide is an alginate.

3. A product according to claim 1, wherein the secondary polymer is an alkali metal carboxymethylcellulose.

4. A product according to claim 1, wherein the secondary polymer is sodium carboxymethylcellulose or potassium carboxymethylcellulose.

5. A product according to claim 1, wherein the sequestrant is a pyrophosphate.

6. A product according to claim 1, wherein the sequestrant is tetrasodium pyrophosphate.

7. A product according to claim 1, wherein the sequestrant is present in an amount of about 0.2 to 0.6% by weight of the oral care composition.

8. A product according to claim 1, wherein the crosslinked outer coating has been crosslinked with a divalent cation.

9. A product according to claim 8, wherein the divalent cation is calcium or zinc.

10. A product according to claim 1, wherein the secondary polymer and the polysaccharide are present in a weight ratio of about 0.8:1 to 1:1.

11. A product according to claim 1 further comprising a humectant selected from the group consisting of glycerin, sorbitol, polyoxyethylene glycol, xylitol, and mixtures thereof.

12. A product according to claim 1 further comprising at least one additional ingredient selected from the group consisting of flavors, foaming agents, salts, sweeteners, preservatives, surfactants, coloring agents, abrasives, thickeners, active agents, and mixtures thereof.

13. A method of making a single unit oral care product comprising:
(a) combining a gellable polysaccharide, a secondary polymer, and a sequestrant to form an oral care composition;
(b) forming at least a portion of the oral care composition into a shaped unit; and
(c) immersing the shaped unit in an aqueous solution of a divalent salt to form a gelled oral care composition.

14. A method of making a single unit oral care product according to claim 13 further comprising:
(d) washing the gelled oral care composition with a second sequestrant.

15. A method of making a single unit oral care product according to claim 13, wherein the second sequestrant is present in an amount of about 0.1 to 1% by weight of the second sequestrant in an aqueous bath.

16. A method of making a single unit oral care product according to claim 13, wherein the gelled oral care composition is rinsed with water prior to or subsequent to being washed with the second sequestrant.

17. A method of making a single unit oral care product according to claim 13, wherein the divalent salt is selected from the group consisting of calcium chloride, zinc lactate, and zinc carbonate.

18. A method of making a single unit oral care product according to claim 13, wherein the oral care composition is deaerated.

19. A single unit oral care product comprising:
an oral care composition comprising an alginate, a sodium carboxymethylcellulose, and a first portion of tetrasodium pyrophosphate,
wherein an outer surface of the oral care composition is encapsulated by an outer coating comprising the alginate crosslinked with a calcium cation, wherein the outer coating has been contacted with a second portion of tetrasodium pyrophosphate.
